(19) United States
(12) Patent Application Publication
(10) Pub. No.: US 2005/0043591 A1
(43) Pub. Date: Feb. 24, 2005

(54) Otoscope

(76) Inventor: Waldemar Witte, Mainz (DE)

Correspondence Address: WILLIAM COLLARD;COLLARD & ROE, P.C., 1077 NORTHERN BOULEVARD, ROSLYN, NY, 11576, US

(21) Appl. No.: 10/496,771

(22) PCT Filed: Nov. 27, 2002
(86) PCT No.: PCT/DE02/04342

(30) Foreign Application Priority Data
Nov. 27, 2001 (DE) 201191873

(51) Int. Cl. A61B 1/267; A61B 1/06

(52) U.S. Cl. 600/200; 600/179

(57) ABSTRACT

An otoscope, preferably for medically examining the human auditory canal, comprising a handle and a head piece, whereby the head piece is connected to an ear funnel, which is provided for partially inserting into an auditory canal, while forming an optical channel therein. The optical channel is terminated on the head piece, specifically, on the side thereof facing away from the ear funnel with an integrated magnifying lens for enabling an enlarged viewing of the auditory canal. A luminous means for illuminating the auditory canal is assigned to the optical channel, whereby the luminous means is at least one light-emitting diode, preferably a white light-emitting diode.

The invention relates to an otoscope, preferably for medically examining the human auditory canal, having a handle piece and a head piece, whereby the head piece is connected with an ear funnel for partial insertion into an auditory canal, including an optical channel that is provided on the side facing away from the ear funnel, with an integrated magnifying lens for enlarged viewing of the auditory canal, whereby a luminous means for illuminating the auditory canal is assigned to the optical channel.

Such an otoscope is known from the international patent application WO 00/51487. The otoscope that is previously known from this international application is characterized, in particular, in that in addition to examining the auditory canal, medications can be injected into the auditory canal, at the same time. In this connection, medicating the auditory canal can preferably take place without having to remove the otoscope. In this way, medicating the auditory canal can be monitored better. In order to be able to both examine the auditory canal and to medicate it, it is necessary to provide the ear funnel that serves for insertion into the auditory canal with a luminous means. In the case of the previously known invention, this is a battery-operated incandescent bulb that is connected with a light guide, in order to be able to emit the light at the desired location within the ear funnel. The light guide offers the advantage, as compared with the incandescent bulb, of easier positioning and, in particular, light deflection within the ear funnel.

The otoscope that has been known for many years to perform ear examinations, also called ear mirror or ear speculum, only guarantees an optimal examination of the auditory canal if the auditory canal is optimally illuminated. In this connection, it has proven to be disadvantageous that the luminous means demonstrate only a fraction of their maximum illumination strength after only a few hours of operation, because of the decreasing battery voltage of the batteries used to supply the luminous means. The treating physician must therefore constantly change the batteries of the otoscope, in order to ensure optimal illumination of the auditory canal, in each instance. In this connection, the batteries have to be disposed of, in some cases, while they are still half charged. This is hardly acceptable, if only for environmental reasons.

The invention is therefore based on the task of creating an otoscope that avoids the disadvantages explained above, and makes optimal illumination of the auditory canal for examination purposes possible at least for a longer period of time.

The task according to the invention is accomplished according to the characteristics of the main claim, in that a light-emitting diode, preferably a white light-emitting diode, is assigned to the ear funnel as the luminous means.

A light-emitting diode is a semiconductor diode that generates light in the conducting direction when it is in operation. In this connection, a semiconductor crystal emits a light signal that is bundled or scattered, respectively, by the lens-type shape of the head of the light-emitting diode. The light generated by means of a light-emitting diode is a colored light, i.e. the light is only emitted over a narrow wavelength range. In contrast to incandescent bulbs, in which an incandescent filament is made to glow, which then emits light, in the case of light-emitting diodes having the semiconductor crystal as mentioned, almost the entire energy used is converted to light. In the case of incandescent bulbs, more than 90° of the energy used is converted to heat and radiated away. Because of almost complete conversion of the energy to light instead of heat, the light generated by a light-emitting diode is called “cool light.”

The higher light yield of the light-emitting diode therefore immediately increases the lifetime of the batteries being used, by many times. Light-emitting diodes furthermore have the advantage of being extremely robust. In contrast to the thin and sensitive incandescent filament of an incandescent bulb, the light-emitting diode is embedded into a plastic lens and therefore decidedly breakage-resistant.

The lifetime of light-emitting diodes is several years in continuous operation.

The use of light-emitting diodes in the sector of ear examinations is particularly successful because it became possible, several years ago, to also produce blue light-emitting diodes. In this way, it became possible to also produce white light-emitting diodes within the course of further development. By means of the combination of blue gallium-nitrite light-emitting diodes and certain pigments, it has been possible for several years now to produce the mixture required for a white light.

Another advantage of the light-emitting diode lies in the emission of diffuse light. In this way, a more uniform illumination of the contours being illuminated is achieved, particularly in the near range.

The otoscope according to the invention, when using white light-emitting diodes, achieves both better illumination of the auditory canal and, because of the greater energy efficiency, lower battery consumption, so that the improved illumination also continues for a longer period of time.

In an advantageous embodiment, a white light-emitting diode is used, the light spectrum of which at least approximately corresponds to the spectrum of sunlight. In this way, natural color reproduction is assured. The correct reproduction of colors is particularly advantageous in diagnosing the auditory canal.

The batteries or rechargeable batteries for operating the light-emitting diode are optimally housed in the handle of the otoscope and can easily be replaced at this location.

The risk of failure of an otoscope operated with a light-emitting diode is essentially restricted to the risk of deep discharge of the rechargeable batteries used, if these are used. In this case, the threshold voltage of the light-emitting diode is dimensioned in such a manner, within the scope of the invention, that it is greater or equal to the final discharge voltage of the power storage being used, so that the risk of deep discharge is also effectively avoided.

In case the light-emitting diode is supplied by a multi-cell rechargeable battery, the final discharge voltage of the rechargeable battery cells and the threshold voltage of the light-emitting diode should be coordinated with one another, taking the cell count into consideration.

In the interests of as long as possible an operating time of the otoscope, nickel/metal hydride rechargeable batteries are preferably used.

In another advantageous embodiment, the light-emitting diode is arranged in the ear funnel in such a manner that the beam of light that exits from the opening of the ear funnel at the insertion tip guarantees optimal illumination of the auditory canal.

In an advantageous embodiment, the head piece is connected with the charging handle of the otoscope in articulated manner. In this way, an optimal position of the otoscope is made possible, in each instance, depending on the patient and depending on the insertion angle and depth.

By using a releasable charging handle, the head piece can simply be set onto a different charging handle if the battery or rechargeable battery voltage decreases, for example in order to be able to continue an ongoing examination. The releasable connection between the otoscope head and the charging handle ideally takes place by way of a simple snap-on adapter.

This adapter can be connected with an otherwise conventional flashlight head, as an alternative to the otoscope head, in order to be able to use the charging handle piece also as a flashlight, if necessary. Expressed the other way around, a flashlight that is appropriately prepared with regard to the power conduction and connected with a connector, but is otherwise conventional, could be fitted with an otoscope head, if necessary. This makes it possible to retrofit an otoscope as a flashlight and vice versa.

The invention will be explained in greater detail below, using an exemplary embodiment shown only schematically in the drawing.

This shows:

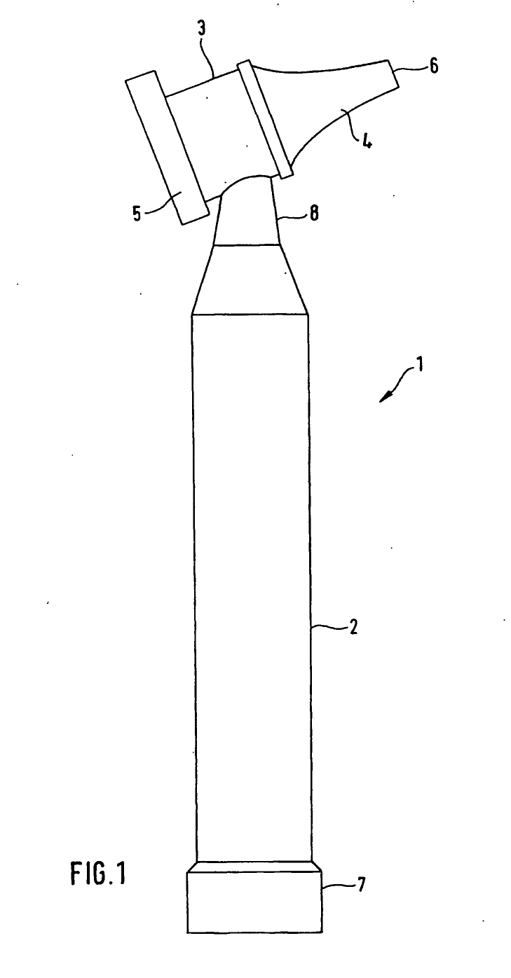

FIG. 1 an otoscope in a side view,

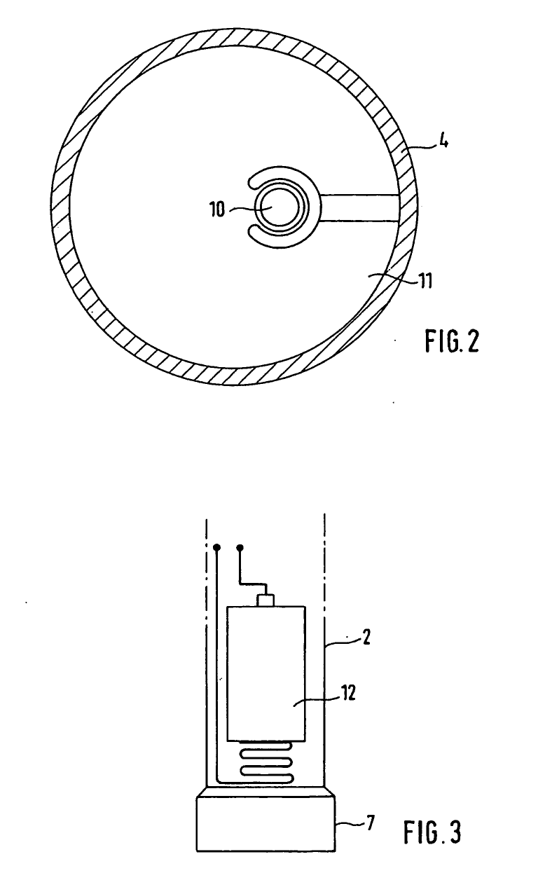

FIG. 2 a detail view regarding the arrangement of the light-emitting diode within an ear funnel, of the otoscope shown in FIG. 1, in cross-section, and

FIG. 3 a detail view regarding the arrangement of a battery within a charging handle, of the otoscope shown in FIG. 1, in cross-section,

FIG. 4 a flashlight having an adapter piece for setting on an otoscope head, in a perspective view,

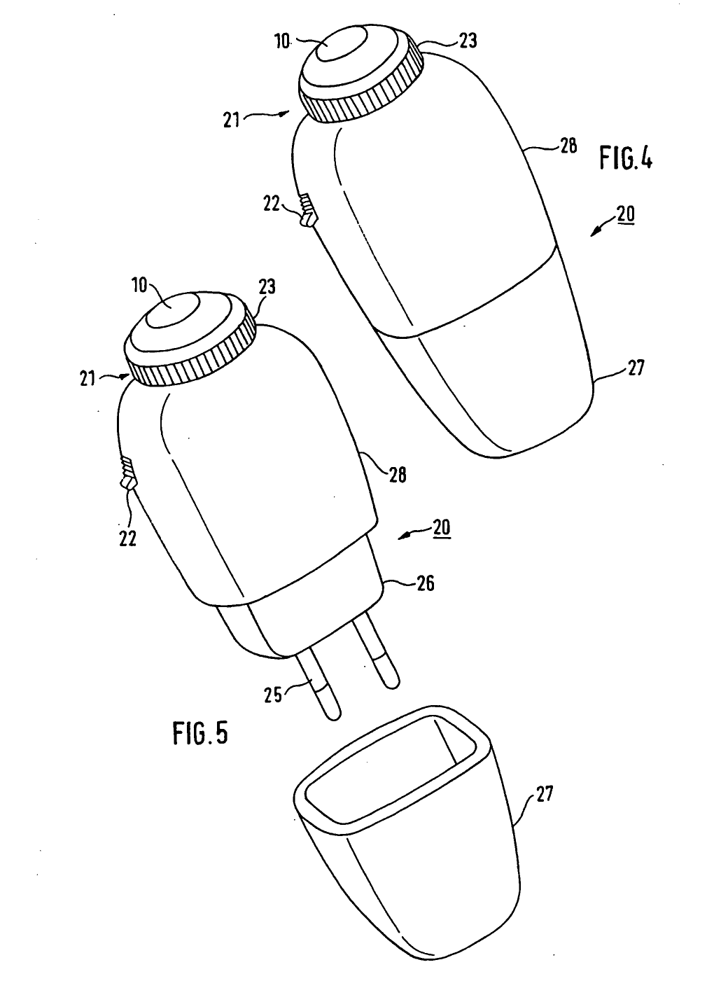

FIG. 5 the flashlight shown in FIG. 4, with the charger sleeve pulled off, in a perspective view, and

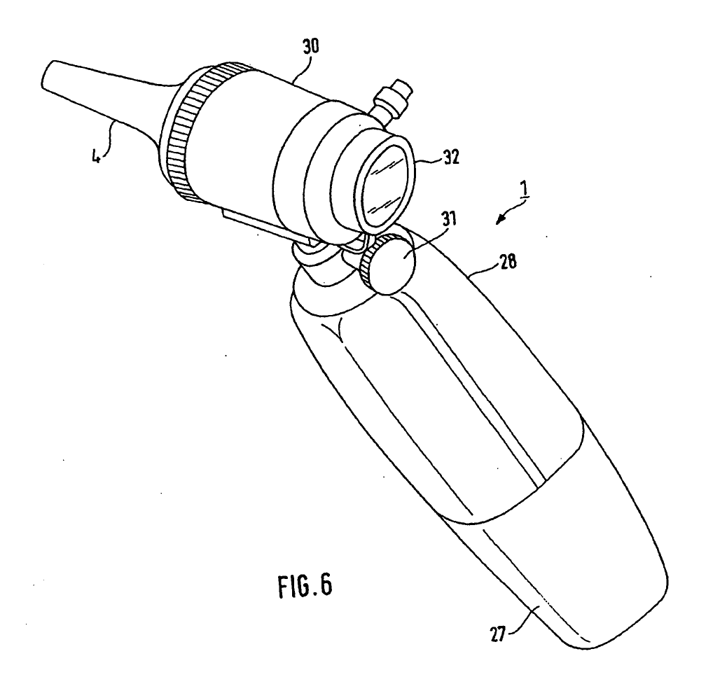

FIG. 6 the flashlight shown in FIG. 4 and 5, with the otoscope head set on.

The otoscope 1 shown in FIG. 1 essentially consists of a charging handle piece 2, a head piece 3, and an ear funnel 4. The ear funnel 4 is either screwed onto the head piece 3, or pressed on by way of a pressure fit. In each case, the ear funnel 4 is releasably connected with the head piece 3, if only for hygiene reasons, and can be replaced or cleaned. The head piece 3 has an integrated magnifying lens 5 at the end removed from the ear funnel 4, which lens represents the end of an optical channel, not shown in detail in FIG. 1, which runs concentrically through the head piece 3, which is configured essentially with rotation symmetry, and through the ear funnel 4, which is also configured essentially with rotation symmetry, and finally opens out at the insertion tip 6. The insertion tip 6 is introduced into the human auditory canal when used for its intended purpose.

The head piece 3 is connected with the charging handle piece 2 in articulated manner, in the embodiment shown in FIG. 1. However, it can also be attached to the charging handle piece 2 in essentially immobile manner.

The special embodiment according to FIG. 2 is a ball joint articulation that sits on a snap-on adapter piece 6. The head piece 3, together with the ear funnel 4, can be pulled off the snap-on adapter 6 in simple manner, and instead, it can be replaced with a flashlight head, for example, the beam direction of which essentially runs in an imaginary axial extension of the charging handle piece 2.

The charging handle piece 2 is closed off with a screw-on closure 7 at its end that faces away from the head piece 3, and is essentially configured as a hollow cylinder to accommodate one or more batteries to supply energy to a luminous means.

The luminous means 10 supplied by the battery interchangeably arranged in the charging handle piece 2 is arranged, in the optical channel of the ear funnel 4 in such a manner, according to FIG. 2, that when it is used for its intended purpose, the most uniform possible illumination of the auditory canal to be examined is obtained. The luminous means is a so-called white light-emitting diode 10 (LED), in which light-emitting diodes that emit blue light, based on gallium nitride, are combined with luminescence pigments to produce a white light diode. The white light-emitting diodes have a light spectrum that is at least similar to sunlight, and thereby guarantee true color reproduction. Light-emitting diodes have a lifetime of clearly more than ten years in continuous operation. Replacement parts are unnecessary during this period, to a great extent. Instead of a thin incandescent filament that is particularly susceptible to failure in the narrowest state, the light-emitting diode is embedded in a clear plastic lens and therefore is considered to be very resistant to breakage. Light-emitting diodes convert more than 90 percent of the energy used into light. The power consumption is therefore only a fraction of the amount of energy otherwise used by incandescent bulbs, for the same light output. In summary, the luminous means does not have to be replaced during the usual lifetime of an otoscope.

The battery 12 arranged in the charging handle piece 2 to supply power, according to FIG. 3, preferably a conventional dry battery, also needs to be replaced only extremely rarely.

According to another embodiment shown in FIG. 4 to 6, the otoscope can also be composed, first of all, of an essentially conventional flashlight 20, the one face of which is the illumination side 21 having a white light-emitting diode 10, which is concentrically arranged within a screw-on adapter piece 23. The flashlight 20 has a center segment 28 into which a rechargeable battery, not shown in detail here, is integrated. The rechargeable battery is connected with the connector of the light-emitting diode 10 and/or its socket by way of a slide switch 22 that is arranged on the side. On the side facing away from the luminous side 21, the center segment 28 is connected with a charging segment. The charging segment consists, according to FIG. 5, essentially of a plug contact 25 for recharging a rechargeable battery, as needed, in a household outlet, not shown here. According to FIG. 4, the plug contact 25 is covered by a plug sleeve 27 that can be pushed onto a connector piece 26 of the plug contact 25, forming a positive lock.

According to the representation in FIG. 6, an otoscope head piece 30 having another integrated white light-emitting diode can be set onto the luminous side of the flashlight 20, onto the screw-on adapter piece, as needed. The otoscope head piece 30 also has an ear funnel 4 for insertion into the auditory canal of a patient.

The additional white light-emitting diode not shown in detail in the drawing, within the otoscope head piece 30, is electrically connected with the battery integrated into the center segment 20, by way of the screw-on adapter. In this connection, the otoscope head piece 30 is firmly screwed onto the screw-on adapter piece by means of a locking screw 31, and thereby secured.

On the side of the otoscope head piece 30 that faces away from the ear funnel 4, a magnifying lens 32 attached in pivoting, articulated manner, is provided, which can be pivoted into the sight channel that opens into the auditory canal of the patient, as needed.

The otoscope head piece 30 thereby represents a practical accessory for the flashlight 20 shown in FIG. 4 and 5.

Above, an otoscope is therefore described, which uses a luminous means that is superior to the state of the art and furthermore demonstrates a low demand for energy.

### Reference Symbol List

1 otoscope
2 charging handle piece
3 head piece
4 ear funnel
5 integrated magnifying lens
6 insertion tip
7 screw-on closure
8 adapter piece
10 light-emitting diode (LED)
11 optical channel
12 battery
20 flashlight
21 luminous side
22 slide switch
23 screw-on adapter piece
25 flashlight
26 connector
27 snap-on sleeve
28 center segment
30 otoscope head piece
31 locking screw
32 magnifying lens
U_{S} threshold voltage of an LED
Uₒ final discharge voltage of a rechargeable battery
z cell count

1. Otoscope, preferably for medically examining the human auditory canal, having a handle piece (2) and a head piece (3), whereby the head piece (3) is connected with an ear funnel (4) for partial insertion into an auditory canal, enclosing an optical channel (11) that ends on the head piece (3), on the side facing away from the ear funnel (4), with an integrated magnifying lens (5) for enlarged viewing of the auditory canal, whereby a luminous means (10) for illuminating the auditory canal is assigned to the optical channel (11),
characterized in that
the luminous means is at least one light-emitting diode (10), preferably a white light-emitting diode.

2. Otoscope according to claim 1, characterized in that the light-emitting diode (10) has a light spectrum that is at least approximately similar to daylight.

3. Otoscope according to claim 2, characterized in that the handle piece (2) of the otoscope is configured as a charging handle (2), in such a manner that at least one battery (12) or at least one rechargeable battery is arranged within the handle piece (2) to supply the voltage of the light-emitting diode (10) assigned to the optical channel (11).

4. Otoscope according to claim 3, characterized in that the rechargeable battery arranged in the handle piece (2) and the light-emitting diode (10) are coordinated with one another in such a manner that the threshold voltage U_{S} of the light-emitting diode (10) is greater than or equal to a final discharge voltage (Uₒ) of the rechargeable battery, in each instance.

5. Otoscope according to claim 3 or 4, characterized in that the rechargeable battery has several cells, whereby the number (z) of the rechargeable battery cells, the final discharge voltage (Uₒ) of these cells, and the threshold voltage (U_{S}) of the light-emitting diode (10) are selected in such a manner that the following applies, in each instance:
U_{S}≧Uₒ×z.

6. Otoscope according to claim 5, characterized in that the rechargeable battery cell(s) (12) is/are nickel/metal hydride rechargeable batteries, in each instance.

7. Otoscope according to one of the preceding claims, characterized in that the light-emitting diode (10) is arranged within the optical channel (11) in such a manner that the distance of the light-emitting diode (10) from the insertion tip (6) of the ear funnel (4) is selected in such a manner that when the otoscope (1) is used for its intended purpose, a uniform illumination of the auditory canal to be examined is obtained.

8. Otoscope according to one of the preceding claims, characterized in that the head piece (3) is connected with the handle piece (2) of the otoscope (1) in articulated manner.

9. Otoscope according to claim 8, characterized in that the head piece (3) is releasably connected with the handle piece (2) of the otoscope (1).

10. Otoscope according to one of the preceding claims, characterized in that the charging handle piece (2) of the otoscope (1) is provided with an adapter piece (23) for snapping on an otoscope head piece (30) and/or a flashlight head.

11. Otoscope according to one of the preceding claims, characterized in that the charging handle piece (2) is provided with a snap-on contact (25), particularly for connecting to a household outlet.
